(19) United States
(12) Patent Application Publication
(10) Pub. No.: US 2018/0296286 A1
(43) Pub. Date: Oct. 18, 2018

(54) VARIABLE SWEEPING FOR INPUT DEVICES

(71) Applicant: Covidien LP, Mansfield, MA (US)

(72) Inventors: William Peine, Ashland, MA (US); Jared Farlow, Cambridge, MA (US); Matthew Blanco, Boston, MA (US)

(21) Appl. No.: 15/766,929

(22) PCT Filed: Oct. 20, 2016
(86) PCT No.: PCT/US2016/057784

§ 371 Date: Apr. 9, 2018

Related U.S. Application Data
(60) Provisional application No. 62/244,762, filed on Oct. 22, 2015.

(51) Int. Cl. A61B 34/37; A61B 18/14; A61B 34/00

(52) U.S. Cl. CPC A61B 34/37; A61B 34/35; A61B 34/76; A61B 18/1442

(57) ABSTRACT

A method for controlling a robotic tool of a robotic surgical system includes pivoting a first control arm of a controller of a user interface of the robotic surgical system with respect to a shaft of the controller and moving a first jaw of the robotic tool of the robotic surgical system a first distance in a first direction relative to a tool axis defined by the robotic tool and moving a second jaw of the robotic tool in response to the pivoting of the first control arm. The second jaw moves the first distance in a second direction opposite the first direction.

### CROSS-REFERENCE TO RELATED APPLICATIONS

This application claims the benefit of and priority to U.S. Provisional Patent Application No. 62/244,762 filed Oct. 22, 2015, the entire disclosure of which is incorporated by reference herein.

### BACKGROUND

Robotic surgical systems have been used in minimally invasive medical procedures. During such a medical procedure, the robotic surgical system is controlled by a surgeon interfacing with a user interface. The user interface allows the surgeon to manipulate an end effector that acts on a patient.

The end effector is inserted into a small incision (via a cannula) or a natural orifice of a patient to position the end effector at a work site within the body of the patient. Some robotic surgical systems include a robotic console supporting a robot arm and at least one end effector such as a scalpel, a forceps, or a grasping tool that is mounted to the robot arm.

Cables may extend from the robot console, through the robot arm, and connect to wrist and/or jaw assemblies of the end effector. In some instances, the cables are actuated by motors that are controlled by a processing system including the user interface for a surgeon or clinician to be able to control the robotic surgical system including the robot arm, the wrist assembly and/or the jaw assembly.

In general, the user interface includes an input controller or handle that is moveable by the surgeon to control the robotic surgical system. Movement of the input controllers and handles is translated to movement of the robotic instruments within the surgical space.

A need exists for input devices with variable sweeping that account for biomechanical factors of users interfacing with robotic surgical systems.

### SUMMARY

The present disclosure generally relates to input devices for robotic surgical systems and methods for controlling the movement of a robotic tool of a robotic surgical system. Specifically, this disclosure is directed to input devices having control arms such that each control arm has a length corresponding to a respective digit of a clinician which engages the respective control arm. By varying the length of the control arms the input devices may account for biomechanical factors of users interfacing with the input device of robotic surgical system. In addition, this disclosure is directed to methods for controlling the movement of a tool in response to control arms of an input device of a robotic surgical system pivoting relative a shaft of the input device. Specifically, the method includes relating an angle between jaws of the tool to an angle between control arms of the input device.

In an aspect of the present disclosure, a method for controlling a robotic tool of a robotic surgical system includes pivoting a first control arm of a controller of a user interface of the robotic surgical system with respect to a shaft of the controller and moving a first jaw of the robotic tool of the robotic surgical system a first distance in a first direction relative to a tool axis defined by the robotic tool and moving a second jaw of the robotic tool in response to the pivoting of the first control arm. The second jaw moves the first distance in a second direction that is opposite the first direction.

In aspects, the user interface transmits a signal in response to pivoting the first control arm. A processing unit of the robotic surgical system may generate a control signal in response to receiving the signal indicative of pivoting the first control arm from the user interface. The processing unit may transmit the control signal to a robotic system to move the first jaw in the first direction and to move the second jaw in the second direction.

In some aspects, pivoting the first control arm with respect to the shaft of the controller includes maintaining a second control arm of the control in position with respect to the shaft. Alternatively, pivoting the first control arm with respect to the shaft of the controller includes pivoting a second control arm of the controller with respect to the shaft. The first control arm and the second control arm may define an arm angle therebetween. The movement of the first jaw the first distance and the movement of the second jaw the second distance may be proportional to a change in the arm angle in response to movement of the first and second control arms.

In certain aspects, pivoting the first control arm with respect to the shaft includes depressing a switch to actuate a function of the robotic tool. Actuating a function of the robotic tool may include ejecting a staple from one of the first or section jaws, delivering electrosurgical energy with the tool, or advancing a knife of the tool. Pivoting the first control arm with respect to the shaft may include receiving tactile feedback in response to abutting the switch before depressing the switch to actuate a function of the tool.

In another aspect of the present disclosure, a robotic surgical system includes a processing unit, a robotic system, and a user interface. The robotic system is in communication with the processing unit. The robotic system includes a robotic tool supported on a shaft that defines a longitudinal tool axis. The robotic tool has first and second jaws movable relative to one another between open and approximated configurations. The first jaw defines a first jaw angle relative to the longitudinal tool axis and the second jaw defines a second jaw angle relative to the longitudinal tool axis. The user interface includes a control that is in communication with the processing unit to manipulate the robotic tool in response to manipulation of the controller. The controller has a controller shaft and first and second control arms. The first and second control arms are pivotally coupled to an end of the shaft. The first control arm defines a first arm angle with the controller shaft and the second control arm defines a second arm angle with the control shaft. Each of the first and second arms is pivotable between open and approximated positions relative to the shaft. The sum of the first and second arm angles is operatively associated with a sum of the first and second jaw angles such that the first and second jaw angles remain equal to one another.

In aspects, the first and second jaws each pivot relative to one another in response to movement of the first arm. Additionally or alternatively, the first and second jaws each pivot relative to one another in response to movement of the second arm.

In some aspects, the first and second jaws remain stationary in response to a change in the first arm angle and a change in the second arm angle. The change in the first arm angle may be a decrease in the first arm angle and the change in the second arm angle may be an increase in the second arm angle such that the decrease in the first arm angle may be equal to the increase in the second arm angle. The robotic system may be configured to actuate a function of the robotic tool when the first and second buttons are depressed.

In certain aspects, the controller includes a first button positioned between the first arm and the control shaft and a second button positioned between the second arm and the control shaft. The first and second buttons may be disposed on the control shaft. The first and second buttons may be configured to provide tactile feedback when the first and second control arms engage the first and second buttons respectively. Alternatively, the first button may be disposed on the first arm and the second button may be disposed on the second arm. The first and second buttons may be configured to provide tactile feedback when the first and second buttons engage the control shaft.

Further details and aspects of exemplary embodiments of the present disclosure are described in more detail below with reference to the appended figures.

### BRIEF DESCRIPTION OF THE DRAWINGS

Various aspects of the present disclosure are described hereinbelow with reference to the drawings, which are incorporated in and constitute a part of this specification, wherein:

FIG. 1 is a schematic illustration of a user interface and a robotic system in accordance with the present disclosure; and

FIG. 2A is a side view of a hand interfacing with a controller of the user interface of FIG. 1, with the controller shown in an open position;

FIG. 2B is a side view of a tool attached to a distal end of one of the linkages of the robotic system in an open configuration corresponding to the open position of the controller of FIG. 2A;

FIG. 3A is the controller of the user interface of FIG. 2A shown in a first approximated position;

FIG. 3B is the tool of FIG. 2B shown in an approximated configuration;

FIG. 4 is the controller of the user interface of FIG. 2A in a second approximated position;

FIG. 5 is a side view of a hand interfacing with another controller of the user interface provided in accordance with the present disclosure; and

FIG. 6 is a schematic diagram of a method for controlling movement of the robotic surgical system of FIG. 1 in accordance with the present disclosure.

### DETAILED DESCRIPTION

Embodiments of the present disclosure are now described in detail with reference to the drawings in which like reference numerals designate identical or corresponding elements in each of the several views. As used herein, the term “clinician” refers to a doctor, a nurse, a surgeon, or any other care provider and may include support personnel. Throughout this description, the term “proximal” refers to the portion of the device or component thereof that is closest to the clinician and the term “distal” refers to the portion of the device or component thereof that is farthest from the clinician.

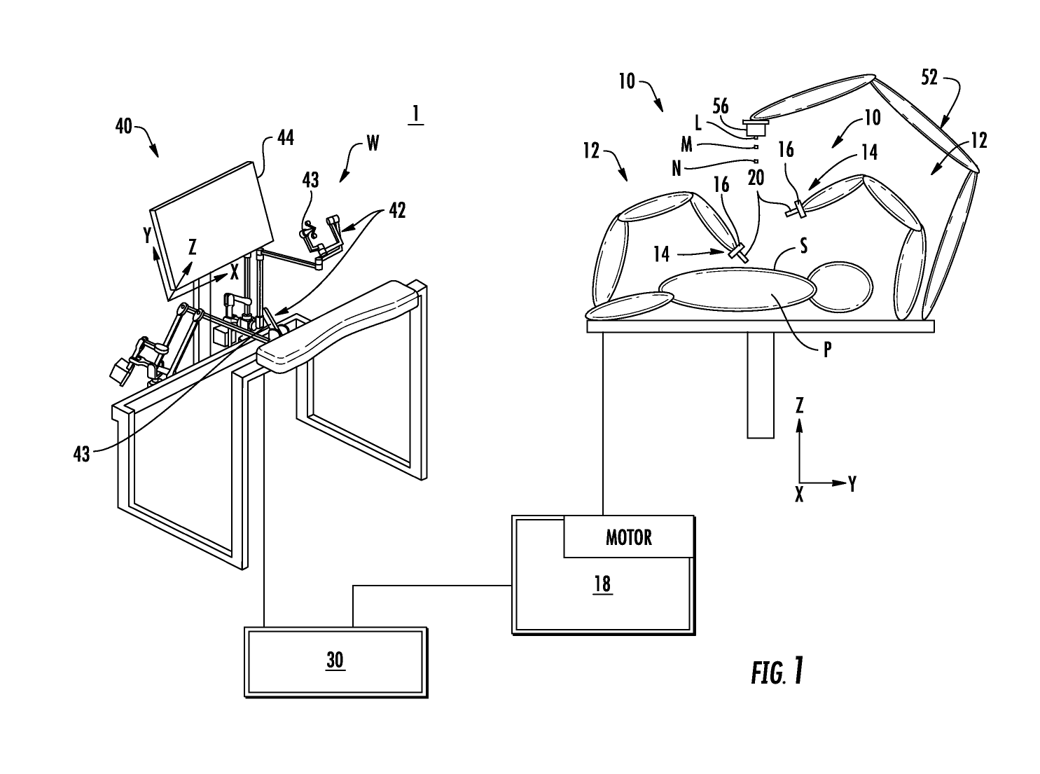

Referring to FIG. 1, a robotic surgical system 1 in accordance with the present disclosure is shown generally as a robotic system 10, a processing unit 30, and a user interface 40. The robotic system 10 generally includes a plurality of arms 12 and a robot base 18. An end 14 of each of the arms 12 supports an end effector or tool 20 which is configured to act on tissue. In addition, the ends 14 of the arms 12 may include an imaging device 16 for imaging a surgical site “S”. The user interface 40 is in communication with robot base 18 through the processing unit 30.

The user interface 40 includes a display device 44 which is configured to display three-dimensional images. The display device 44 displays three-dimensional images of the surgical site “S” which may include data captured by imaging devices 16 positioned on the ends 14 of the arms 12 and/or include data captured by imaging devices that are positioned about the surgical theater (e.g., an imaging device positioned within the surgical site “S”, an imaging device positioned adjacent the patient “P”, imaging device 56 positioned at a distal end of an imaging arm 52). The imaging devices (e.g., imaging devices 16, 56) may capture visual images, infra-red images, ultrasound images, X-ray images, thermal images, and/or any other known real-time images of the surgical site “S”. The imaging devices transmit captured imaging data to the processing unit 30 which creates three-dimensional images of the surgical site “S” in real-time from the imaging data and transmits the three-dimensional images to the display device 44 for display.

The user interface 40 also includes input handles 42 which allow a clinician to manipulate the robotic system 10 (e.g., move the arms 12, the ends 14 of the arms 12, and/or the tools 20). Each of the input handles 42 is in communication with the processing unit 30 to transmit control signals thereto and to receive feedback signals therefrom. Additionally or alternatively, each of the input handles 42 may include control interfaces (not shown) which allow the surgeon to manipulate (e.g., clamp, grasp, fire, open, close, rotate, thrust, slice, etc.) the tools 20 supported at the ends 14 of the arms 12.

Each of the input handles 42 is moveable through a predefined three-dimensional workspace to move the ends 14 of the arms 12 within a surgical site “S”. The three-dimensional images on the display device 44 are orientated such that the movement of the input handle 42 moves the ends 14 of the arms 12 as viewed on the display device 44. It will be appreciated that the orientation of the three-dimensional images on the display device may be mirrored or rotated relative to view from above the patient “P”. In addition, it will be appreciated that the size of the three-dimensional images on the display device 44 may be scaled to be larger or smaller than the actual structures of the surgical site permitting the surgeon to have a better view of structures within the surgical site “S”. As the input handles 42 are moved, the tools 20 are moved within the surgical site “S” as detailed below. As detailed herein, movement of the tools 20 may also include the ends 14 of the arms 12 which support the tools 20.

For a detailed discussion of the construction and operation of a robotic surgical system 1, reference may be made to U.S. Pat. No. 8,828,023 the entire contents of which are incorporated herein by reference.

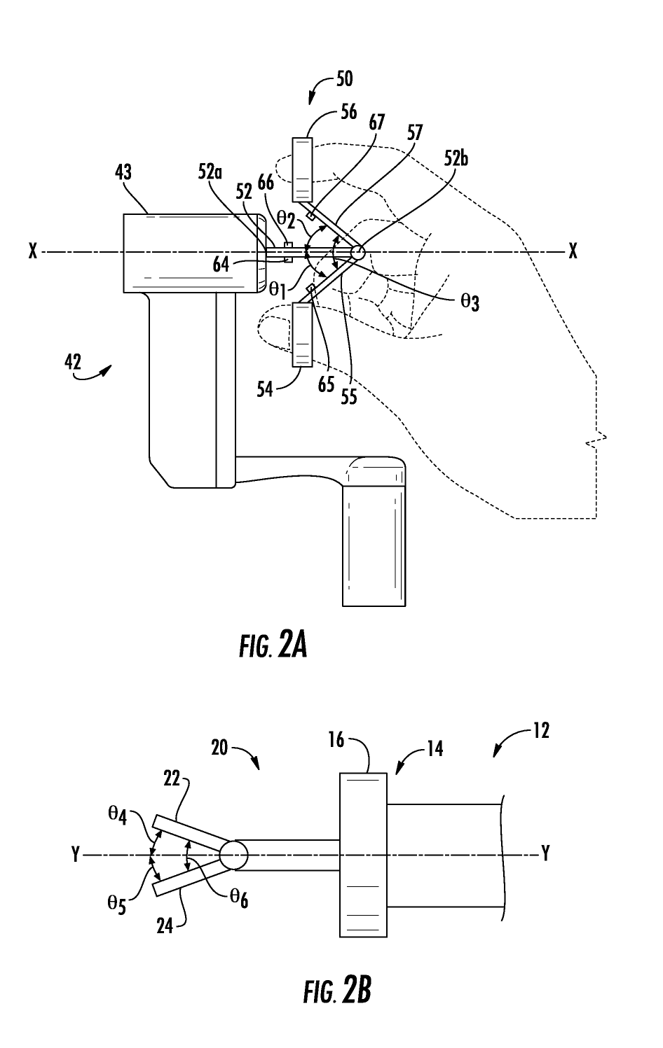

With reference to FIG. 2A, each input handle 42 includes a controller 50 for manipulating a respective tool 20 and a respective arm 12. The controller 50 includes a shaft 52, a thumb loop 54, and a finger loop 56. The shaft 52 has a first end 52a that is selectively coupled to the input handle 42 and a second end 52b. The shaft 52 defines an axis “X-X” between the first and second ends 52a, 52b. The thumb loop 54 is coupled to the second end 52b of the shaft 52 by a control arm 55 and the finger loop 56 is coupled to the second end 53b by a control arm 57. The control arms 55, 57 are pivotable in a plane orthogonal to the axis “X-X” of the shaft 52. The plane may pass through the axis “X-X” or be offset from the axis “X-X”.

The control arm 55 that supports the thumb loop 54 defines an angle “θ₁” with the axis “X-X” within the plane and the control arm 56 that supports the finger loop 56 defines an angle “θ₂” with the axis “X-X” within the plane. In addition, an angle “θ₃”, which is the sum of angle “θ₁” and angle “θ₂”, is defined between the first and second control arms 55, 57. The angles “θ₁”, “θ₂”, “θ₃” are changed as the loops 54, 56 are moved or swept within the plane towards and away from the axis “X-X”.

With additional reference to FIG. 2B, the controller 50 may be associated with a tool 20 having first and second jaws 22, 24. The first and second jaws 22, 24 are moveable relative to one another between an open configuration and a closed configuration. In the open configuration, the first and second jaws 22, 24 are spaced-apart from one another and in the closed configuration, the first and second jaws 22, 24 are approximated relative to one another. In the closed configuration, the first and second jaws 22, 24 may cooperate to grasp tissue and/or tools therebetween.

The tool 20 defines an axis “Y-Y” that passes between the first and second jaws 22, 24. The first jaw 22 defines an angle “θ₄” with the axis “Y-Y” and the second jaw 24 defines an angle “θ₅” with the axis “Y-Y”. In addition, an angle “θ₆”, which is the sum of angle “θ₄” and angle “θ₅”, is defined between the first and second jaws 22, 24.

The controller 50 is operatively associated with the tool 20 through the user interface 40 and the processing unit 30. The first and second jaws 22, 24 are operatively associated with the first and second control arms 55, 57 such that movement of the control arms 55, 57 relative to the axis “X-X” effects movement of the first and second jaws 22, 24 relative to the axis “Y-Y”.

In embodiments, the first control arm 55 is associated with the first jaw 22 such that the angle “θ₁” of the first control arm 55 with the axis “X-X” is associated with the angle “θ₄” of the first jaw 22 with the axis “Y-Y” such that changes in the angle “θ₁” effect changes in the angle “θ₄”. In addition, the second control arm 57 is associated with the second jaw 24 such that the angle “θ₂” between the second control arm 57 and the axis “X-X” is associated with the angle “θ₅” between the second jaw 24 and the axis “Y-Y” such that changes in the angle “θ₂” effect changes in the angle “θ₅”.

Changes in the angle “θ₁” may be scaled to changes in the angle “θ₄” by a first scaling factor “SF₁” and changes in the angle “θ₂” may be scaled to changes in the angle “θ₅” by a second scaling factor “SF₂”. The first and second scaling factors “SF₁”, “SF₂” may be determined by the anatomical features of the clinician.

For example, movement of the first control arm 55 is effected by movement of the thumb loop 54 that is engaged by the thumb of a clinician and the first scaling factor “SF₁” may be scaled relative to the movement of the thumb of a clinician from a closed position, where the thumb is adjacent or in contact with the shaft 52, to a fully extended position, where the thumb is extended away from the shaft 52. Similarly, movement of the second control arm 57 is effected by movement of the finger loop 56 that is engaged by the index finger of a clinician and the second scaling factor “SF₂” may be scaled relative to the movement of the index finger of a clinician from a closed position, where the index finger is adjacent or in contact with the shaft 52, to a fully extended position, where the index finger is extended away from the shaft 52. In such embodiments, the first and second scaling factors “SF₁”, “SF₂” are calibrated such that movement of the thumb of the clinician between the closed position and the extended position effects a change in the angle “θ₄” of the first jaw 52 that is equal to the change in the angle “θ₅” of the second jaw 54 when the index finger is moved between the closed position and the extended position. It will be appreciated that in such a configuration, movement of the first jaw 52 is independent of movement of the second jaw 54. It is contemplated, that the first and second scaling factors “SF₁”, “SF₂” may be set during manufacturing of controller 50, may be set by a central system of the medical facility based on a clinician using the surgical system 1, or may be set by a calibration routine before the start of a procedure by measuring the movements of a clinician using the surgical system 1.

In some embodiments, the first control arm 55 is associated with the first jaw 22 and the second control arm 57 is associated with the second jaw 24 such that changes in the angle “θ₃”, defined between the first and second control arms 55, 57, effects changes in the angle “θ₆”, defined between the first and second jaws 22, 24.

Changes in the angle “θ₃” may be scaled to changes in the angle “θ₆” by a third scaling factor “SF₃”. For example, the movement of the control arms 55, 57 may be scaled down such that a change of 30° of the angle “θ₃” between the control arms 55, 57 may result in a change of 15° in angle “θ₆” between the first and second jaws 22, 24. It is also contemplated that the movement of the control arms 55, 57 may be scaled up such that a change of 15° of the angle “θ₃” between the control arms 55, 57 may result in a change of 30° in angle “θ₆” between the first and second jaws 22, 24. It will be appreciated that in such embodiments, movement of the first and second jaws 22, 24 is related to one another. It is within the scope of this disclosure that one of the first or second jaws 22, 24 may be fixed relative to the axis “Y-Y” such that changes in the angle “θ₃” between control arms 55, 57 effect movement of only one of the first or second jaws 22, 24 based on the change in the angle “θ₃”. Such embodiments may be advantageous when one jaw (e.g., second jaw 24) of the tool 20 has a stationary jaw and the other jaw (e.g., the first jaw) is moveable relative to the stationary jaw to transition the jaws between the open and closed configurations; for example, when the tool 20 is a stapling instrument.

In some embodiments, a control axis (not explicitly shown) passes through the second end 52b of the shaft 52, defines an angle with the axis X-X in the plane, and passes between the control arms 55, 57. In such embodiments, the angle θ₁ is defined between the control arm 55 and the control axis and the angle θ₂ is defined between the control arm 57 and the control axis. By defining the angles θ₁ and θ₂ relative to the control axis, the movement of the control arms 55, 57 may correspond to the anatomical features of the clinician. In particular embodiments, the control axis may be aligned with one of the control arms 55, 57 such that a respective one of the angles θ₁ and θ₂ may be substantially 0° to represent a tool 20 with a stationary jaw (e.g., a stapling instrument) such that movement of either control arm 55, 57 moves the non-stationary jaw relative to the stationary jaw.

In some embodiments, a tool axis (not explicitly shown) passes through a pivot point between the first and second jaws 22, 24 of the tool 20, defines an angle with the axis Y-Y, and passes between the first and second jaws 22, 24. In such embodiments, the angle θ₄ is defined between the first jaw 22 and the tool axis and the angle θ₅ is defined between the second jaw 24 and the control axis. By defining the angles θ₄ and θ₅ relative to the tool axis, the movement of the first and second jaws 22, 24 may correspond to the anatomical features of the clinician. It is contemplated that the tool axis may define an angle with the axis Y-Y that is similar to an angle defined between the control axis and the axis X-X.

Referring back to FIG. 2A, the controller 50 includes an activation switch assembly including one or more activation switches (e.g., switches 64, 65, 66, 67) to activate a function of the tool 20. Examples of such functions include, but are not limited to, firing a fastener from one of the first or second jaws 22, 24 of the tool 20, advancing a knife (not shown) positioned in one of the first or second jaws 22, 24, delivering electrosurgical energy to tissue with the tool 20, or any combinations thereof. The activation switch assembly includes a switch 64 positioned on the shaft 52 between the shaft 52 and the control arm 55, a switch 65 positioned on the control arm 55, a switch 66 positioned on the shaft 52 between the shaft 52 and the control arm 57, and a switch 67 positioned on the control arm 57. As shown, the activation switch assembly includes two pairs of switches, switches 64 and 66 and switches 65 and 67; however, it is contemplated that the activation switch assembly may include a single pair of switches.

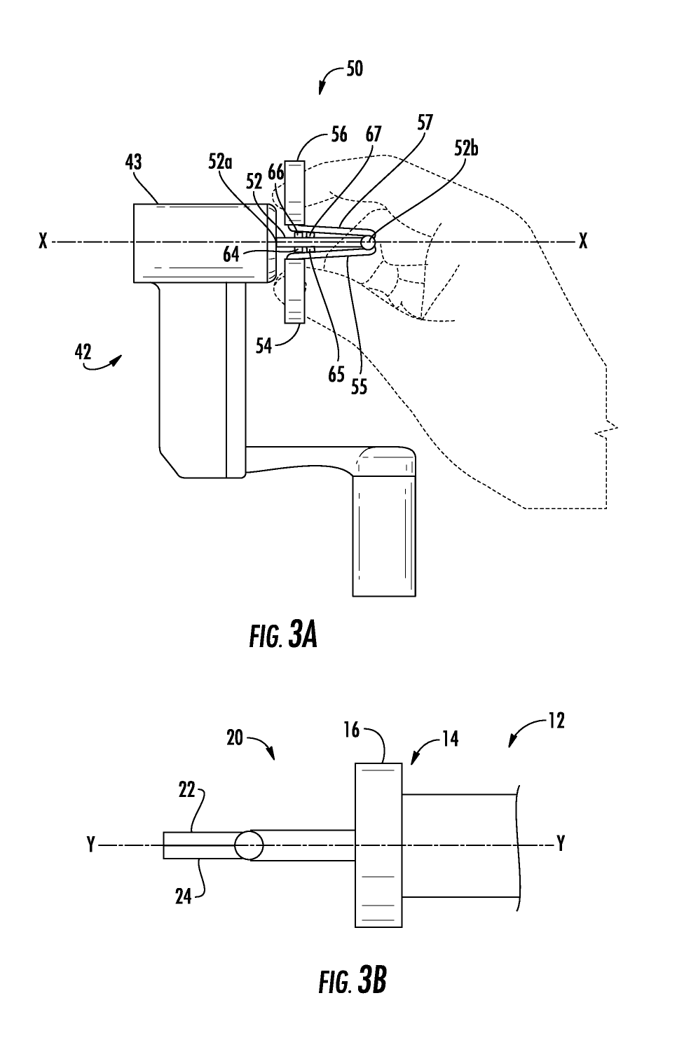

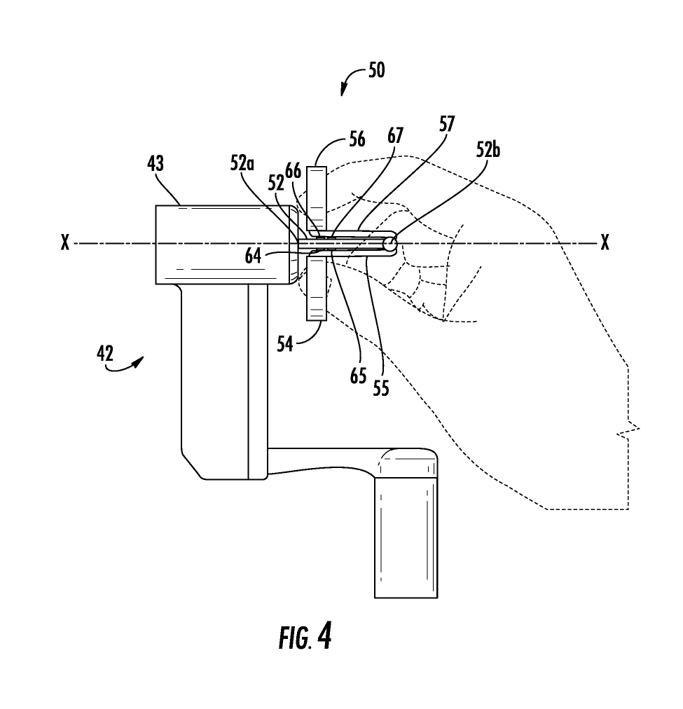

Referring now to FIGS. 2A-4, the control arms 55, 57 are moveable between an open position (FIG. 2A), a first approximated position (FIG. 3A), and a second approximated position (FIG. 4) and first and second jaws 22, 24 of the tool 20 are moveable between an open configuration (FIG. 2B) and an approximated configuration (FIG. 3B) in response to movement of the control arms 55, 57.

Initially and with particular reference to FIGS. 2A and 2B, the control arms 55, 57 are in the open position, the first and second jaws 22, 24 are in the open configuration, the switches 64-67 are in an unactuated position, and the first and second jaws 22, 24 of the tool 20 in the open configuration such that the first and second jaws 22, 24 are spaced apart from one another.

When the control arms 55, 57 are in the first approximated position, the control arms 55, 57 abut the switches 64, 66 positioned on the shaft 52, the switches 65, 67 positioned on the control arms 55, 57 abut the shaft 52, and the first and second jaws 22, 24 of the tool 20 are in the approximated configuration. The switches 64-67 are biased to the unactuated position such that each of the switches 64-67 provides tactile feedback when the switches 64-67 abut the shaft 52 or are abutted by the control arms 55, 57, respectively. It will be appreciated that the tactile feedback of the switches 64-67 may prevent in advertent actuation of the switches 64-67.

When the control arms 55, 57 move from the first approximated position to the second approximated position, the control arms 55, 57 depress switches 64, 66 to the actuated position and the switches 65, 67 engage the shaft 52 to depress to the actuated position, and the first and second jaws 22, 24 of the tool 20 remain in the approximated configuration. As the switches 64-67 are moved to the actuated position, a function associated with each switch 64-67 or each pair of switches (e.g., switches 64 and 66 or switches 65 and 67) is activated such that the tool 20 performs a desired function, as detailed above.

In an aspect of the present disclosure, the controller 50 is manipulated to grasp and release tissue with the first and second jaws 22, 24 of the tool 20 until a desired portion of the tissue is grasped between the first and second jaws 22, 24. Then, the controller 50 is manipulated such that the tool 20 completes a desired function to the desired portion of the tissue. Specifically, the thumb loop 54 and the finger loop 56 are manipulated to move the control shafts 55, 57 between the open and first approximated position to move the first and second jaws 22, 24 between the open and approximated configurations to grasp, release, and reposition tissue. When the first and second jaws 22, 24 are in the approximated configuration with a desired portion of tissue therebetween, the thumb loop 54 and the finger loop 56 are manipulated to move the control shafts 55, 57 from the first approximated configuration to the second approximated configuration such that the switches 64-67 are depressed or moved to the actuated position. As the switches 64-67 reach the actuated position, electrosurgical energy is delivered to the desired portion of tissue with the tool 20.

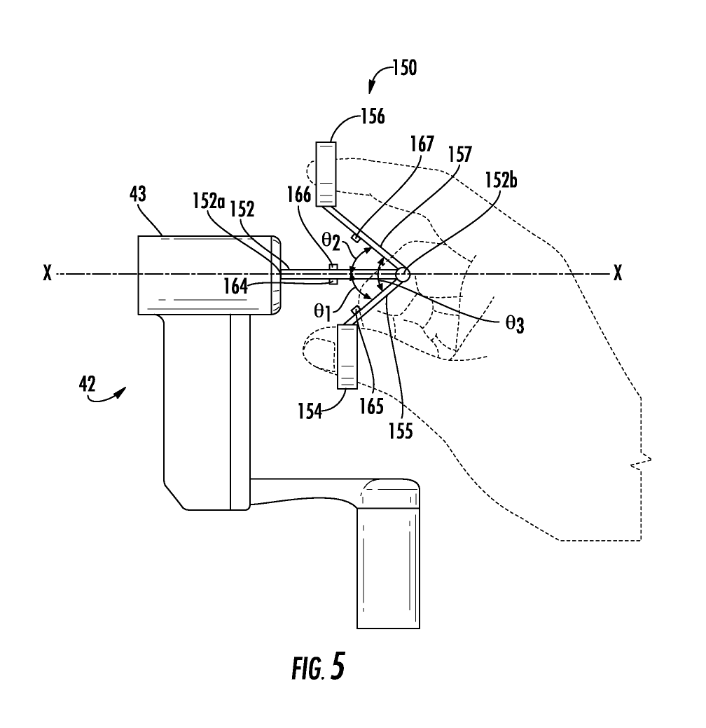

Referring now to FIG. 5, another controller 150 is provided in accordance with the present disclosure. The controller 150 is substantially similar to the controller 50 detailed above as such for brevity only the differences will be detailed herein. The controller 150 includes a shaft 152, a thumb loop 154, and a finger loop 156. The thumb loop 154 is coupled to the second end 152b of the shaft 152 by a control arm 155 having a first length and the finger loop 156 is coupled to the second end 152b by a control arm 157 having a second length. The second length is greater than the first length to compensate for anatomical differences in the length of a finger (e.g., an index finger) of a clinician and a thumb of a clinician. The difference in the first and second lengths requires the finger loop 156 to sweep a greater arc towards or away from the shaft 152 to effect a change in the angle “θ₂” than an arc swept by the thumb loop 154 towards or away from the shaft 152 to effect an equal change in the angle “θ₁”.

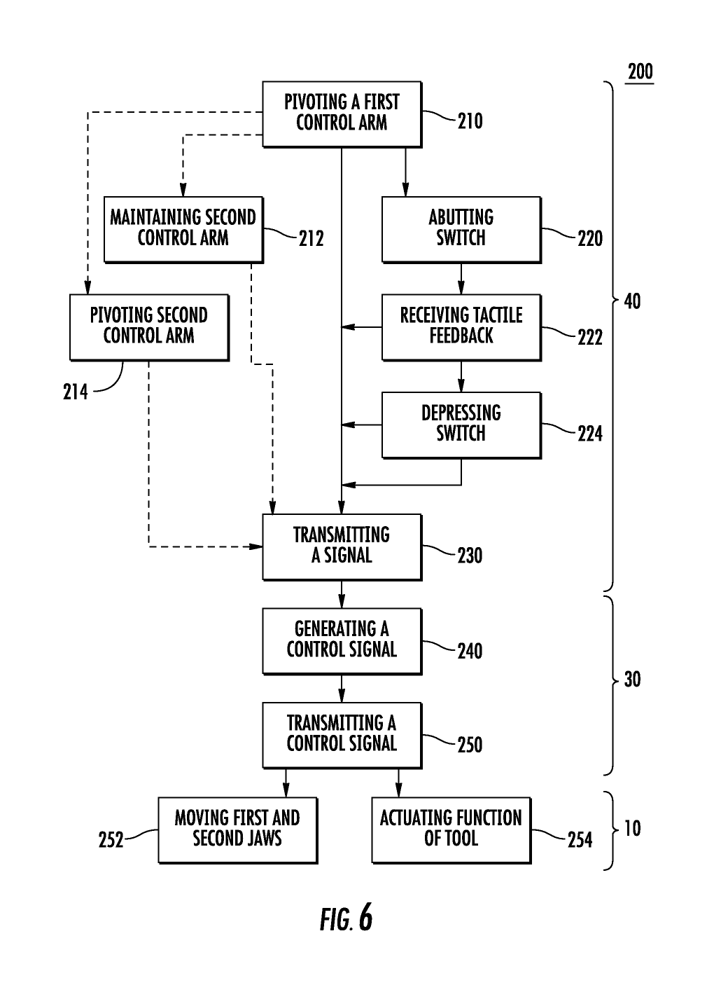

Referring now to FIG. 6, a method 200 of controlling a robotic tool of a robotic surgical system is described in accordance with the present disclosure. Initially, a first control arm (e.g., control arm 57, 157) of a user interface 40 is pivoted or swept towards or away from a shaft pivotally supporting the control arm (e.g., shaft 52, 152) (Step 210). While the first control arm is pivoted, a second control arm (e.g., control arm 55, 155) is either maintained in position such that an angle between the second control arm and the shaft is maintained (Step 212) or the second control arm is also pivoted towards or away from the shaft (Step 214). In response to pivoting the first control arm and/or the second control arm, the user interface 40 transmits a signal to a processing unit 30 indicative of a change in an angle “θ₃” defined between the first and second control arms (Step 230).

In response to the signal from the user interface 40, the processing unit 30 generates a control signal (Step 240). The processing unit 30 transmits the control signal to a robotic system 10 (Step 250). In response to the control signal, the robotic system 10 moves first and second jaws relative to one another such that an angle “θ₆” defined between the first and second jaws of the robotic system changes proportional to the change in the angle “θ₃” (Step 252).

When the first or second control arms are pivoted, the control arm may abut a switch (e.g., switch 64-67) (Step 220) such that tactile feedback is received through a loop (e.g., thumb loop 54, 154 or finger loop 56, 156) (Step 222). After the tactile feedback is received, subsequent pivoting of the control arm towards the shaft depresses the switch (Step 224). In such instances, signal transmitted by the user interface (Step 230) is indicative of the button being depressed, such that the control signal generated and transmitted by the processing unit (Steps 240, 250) actuates a function of the robotic tool of the robotic system (Step 254). It is contemplated that pivoting the first control arm may first move the first and second jaws an angle “θ₆” and then actuate a function of the robotic tool.

The user interface 40 and the processing unit 30 may generate and transmit the signal and control signal, respectively, in a wired or wireless manner. Such wireless connections detailed herein (e.g., between controller 63 and the processing unit 30) may be via radio frequency, optical, WIFI, Bluetooth® (an open wireless protocol for exchanging data over short distances (using short length radio waves) from fixed and mobile devices, creating personal area networks (PANs)), ZigBee® (a specification for a suite of high level communication protocols using small, low-power digital radios based on the IEEE 802.15.4-2003 standard for wireless personal area networks (WPANs)), etc.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed:

1. A method of controlling a robotic tool of a robotic surgical system, the method comprising:
pivoting a first control arm of a controller of a user interface of the robotic surgical system with respect to a shaft of the controller; and
moving a first jaw of a robotic tool of the robotic surgical system a first distance in a first direction relative to a tool axis defined by the robotic tool and moving a second jaw of the robotic tool, the first distance, in a second direction opposite the first direction in response to the pivoting of the first control arm.

2. The method according to claim 1, further comprising transmitting a signal in response to pivoting of the first control arm.

3. The method according to claim 2, further comprising:
generating a control signal within a processing unit in response to receiving the signal indicative of pivoting the first control arm; and
transmitting the control signal to a robotic system to move the first jaw in the first direction and to move the second jaw in the second direction.

4. The method according to claim 1, wherein pivoting the first control arm with respect to the shaft of the controller includes maintaining a second control arm of the controller in position with respect to the shaft.

5. The method according to claim 1, wherein pivoting the first control arm with respect to the shaft of the controller includes pivoting a second control arm of the controller with respect to the shaft, the first control arm and the second control arm defining an arm angle therebetween, and wherein moving the first jaw the first distance and moving the second jaw the second distance is proportional to a change in the arm angle in response to moving the first and second control arms.

6. The method according to claim 1, wherein pivoting the first control arm with respect to the shaft includes depressing a switch to actuate a function of the robotic tool.

7. The method according to claim 6, wherein actuating a function of the robotic tool includes at least one of ejecting a staple from one of the first or second jaws, delivering electrosurgical energy with the tool, or advancing a knife of the tool.

8. The method according to claim 6, wherein pivoting the first control arm with respect to the shaft includes receiving tactile feedback in response to abutting the switch before depressing the switch to actuate a function of the tool.

9. A robotic surgical system comprising:
a processing unit;
a robotic system in communication with the processing unit and including a robotic tool supported on a shaft that defines a longitudinal tool axis, the robotic tool having first and second jaws moveable relative to one another between an open configuration and an approximated configuration, the first jaw defining a first jaw angle relative to the longitudinal tool axis and the second jaw defining a second jaw angle relative to the longitudinal tool axis;
a user interface including a controller and being in communication with the processing unit to manipulate the robotic tool of the robotic system in response to manipulation of the controller, the controller having a controller shaft, a first control arm, and a second control arm, the first and second control arms pivotally coupled to an end of the shaft, the first control arm defining a first arm angle with the controller shaft and the second control arm defining a second arm angle with the control shaft, the first and second control arms each pivotable between an open position and an approximated position relative to the shaft, wherein a sum of the first and second arm angles is operatively associated with a sum of the first and second jaw angles such that the first and second jaw angles remain equal to one another.

10. The robotic surgical system according to claim 9, wherein the first and second jaws each pivot relative to one another in response to movement of the first arm.

11. The robotic surgical system according to claim 9, wherein the first and second jaws each pivot relative to one another in response to movement of the second arm.

12. The robotic surgical system according to claim 9, wherein the first and second jaws remain stationary in response to a change in the first arm angle and a change in the second arm angle.

13. The robotic surgical system according to claim 12, wherein the change in the first arm angle is a decrease in the first arm angle and the change in the second arm angle is an increase in the second arm angle.

14. The robotic surgical system according to claim 13, wherein the decrease in the first arm angle is equal to the increase in the second arm angle.

15. The robotic surgical system according to claim 9, wherein the controller includes a first button positioned between the first arm and the control shaft and a second button positioned between the second arm and the control shaft, and wherein the robotic system is configured to actuate a function of the robotic tool when the first and second buttons are depressed.

16. The robotic surgical system according to claim 15, wherein the first and second buttons are disposed on the control shaft.

17. The robotic surgical system according to claim 16, wherein at least one of the first and second buttons are configured to provide tactile feedback when the first and second control arms engage the first and second buttons, respectively.

18. The robotic surgical system according to claim 15, wherein the first button is disposed on the first arm and the second button is disposed on the second arm.

19. The robotic surgical system according to claim 18, wherein at least one of the first and second buttons are configured to provide tactile feedback when the first and second buttons engage the control shaft.
